(19) United States
(12) Patent Application Publication
(10) Pub. No.: US 2009/0117090 A1
(43) Pub. Date: May 7, 2009

(54) INHIBITION OF GLYCOGEN SYNTHASE KINASE 3 BETA IN ARTERIAL REPAIR AND STENT RE-ENDOTHELIALIZATION

(75) Inventors: Edward R.M. O'BRIEN, Ottawa (CA); Benjamin HIBBERT, Ottawa (CA); Xiaoli MA, Nepean (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: OTTAWA HEART INSTITUTE RESEARCH CORPORATION, Ottawa, ON (CA)

(21) Appl. No.: 12/263,870

(22) Filed: Nov. 3, 2008

Related U.S. Application Data
(60) Provisional application No. 60/984,607, filed on Nov. 1, 2007.

(51) Int. Cl. A61F 2/00; A61K 38/45; A61P 9/00

(52) U.S. Cl. 424/94.5; 424/423

(57) ABSTRACT

A device having a kinase inhibitor for treating or preventing vascular disease in a subject and a method of treating a subject therewith, is disclosed. The device can be a stent coated with the kinase inhibitor. The kinase inhibitor can be a glycogen synthase kinase (GSK) inhibitor, such as a GSK-3β inhibitor. Coronary artery disease and ischemic heart disease can be treated.

### CROSS-REFERENCE TO RELATED APPLICATIONS

The present application claims the benefit of priority of U.S. Provisional patent application Ser. No. 60/984,607, filed Nov. 1, 2007, and incorporated herein by reference.

### FIELD OF THE INVENTION

The present invention relates generally to a method and device for treating and preventing cardiovascular disease. More particularly, the present invention relates to a method and device for treating vascular disease.

### BACKGROUND OF THE INVENTION

Revascularization of the coronary circulation by percutaneous intervention has become the preferred strategy in patients with ischemic heart disease (1). Development of bare metal and drug eluting stents have reduced revascularization rates when compared to balloon angioplasty (2). Current strategies for reducing in-stent neointima formation exploit the anti-proliferative and anti-inflammatory effects of paclitaxel and sirolimus on vascular smooth muscle cells. However, the actions of these drugs and/or the polymers within which they are embedded may, in some instances, impede homeostatic healing thus resulting in the genesis of incompletely re-endothelialization stented vascular segments that are at risk for sudden thrombosis—a potentially life threatening and difficult to predict hazard (3).

Recent developments have led to the understanding that circulating progenitor populations, specifically endothelial progenitor cells, play key roles in arterial repair following injury. For example, estrogen (4), statins (5), granulocyte colony stimulating factor (6), and direct transplantation of ex vivo cultured EPCs (7) have been shown to improve cell mediated repair and ultimately reduce neointima formation in animal models. Clinically, transplantation of patient cells represents an attractive strategy for enhancing vessel re-endothelialization and in some studies this technology has been shown to result in an improvement in left ventricular ejection fraction (8) and survival (9) post myocardial infarction. However, one major limiting aspect of this approach is that EPCs from CAD patients are low in abundance (10) and functionally incompetent (11).

In recent years it has become increasingly apparent that EPCs help mediate arterial repair following mechanical injury (6; 7). Relative to healthy controls, patients with CAD have a paucity of circulating EPCs (10)—an observation that led researchers to test the hypothesis that increasing EPC numbers would lead to improved arterial repair (28). However, it soon became obvious that the in vitro characteristics or qualitative properties of EPCs were equally important (19; 29) and that increasing the number of dysfunctional cells would be insufficient for therapeutic purposes (11). Transplantation of ex vivo manipulated cells is both impractical and labor intensive, and often with very modest EPC yields because of the metabolic and/or genetic profile of the patient (29). Ultimately, pharmacologic enhancement of this endogenous repair system is the most likely manner in which EPC biology will be used to improve arterial repair.

Glycogen synthase kinase 3β is a serine/threonine protein kinase known to negatively regulate Wnt signaling through phosphorylation of the nuclear transcription factor β-catenin and hence, direct its degradation (30). Recently, GSK 3 inhibition was shown to stimulate progenitor and hematopoietic stem cell capacity in vivo through modulation of Wnt, Hedgehog and Notch signaling (14). Moreover, transfection of progenitor cells with an inactive GSK-3β has been shown to improve the angiogenic profile EPCs (16). Finally, Wnt signaling has been implicated in maintaining progenitor cell pluropotency and differentiation with studies showing that GSK-3β inhibition can facilitate transdifferentiation into vascular cell and cardiomyocyte lineages (31; 32).

In general, the current commercially available drug coated stents are thought to attenuate in-stent restenosis by anti-proliferative and anti-inflammatory mechanisms (1; 33). To a large extent, the development of these agents reflects the perspective that neointimal formation is largely due to medial smooth muscle cell proliferation following vascular injury. It is only recently that the role of endothelial progenitor cells has become apparent in reducing vessel narrowing following mechanical insult. EPCs are thought to participate in the protective process of re-endothelialization. However, vascular progenitor cells akin to smooth muscle cells are also thought to be instrumental in neointimal formation (34-38), although the degree to which they contribute to vascular lesions continues to be debated (39; 40). The involvement of these vascular precursor cells may help explain why the contribution of cell proliferation in restenotic lesions after vascular interventions involving balloon angioplasty and/or stent insertion may be less than was initially anticipated. Irrespective of the origin of the cells, it is established that current DES-based anti-proliferative therapies curtail SMC accumulation within stents but also cause marked delays in re-endothelialization and expose the patient to the risk of stent thrombosis (42).

In vivo, the time course of endothelial healing after stent implantation varies in different models. For example, Virmani's group found that deployment of DESs in the iliac arteries of pigs and rabbits resulted in complete endothelialization by 14 and 21 days respectively (43). In humans, time to re-endothelialization is not clear although our best evidence suggests that with bare metal stents it occurs by the 3 to 4 months post deployment (44; 45) and with DESs much later (46). Acceleration of re-endothelialization is thought to contribute to stabilization of neointimal development ultimately resulting in attenuation of vessel re-narrowing (47-49). While it may be unclear if re-endothelialization alone is important for preventing neointima formation, in at least two studies re-endothelialization has been highlighted as a critical event in preventing adverse clinical outcomes such as late stent thrombosis (44; 50).

Klugherz et al. (51) found with sirolimus eluting stents a 45% reduction in neointimal area in the same rabbit model of stent neointimal formation. Furthermore, studies in rabbits with paclitaxel-coated stents resulted in a 48% reduction in neointimal thickness when compared to control stents (52).

Glycogen synthase kinase 3, originally described for its function of inactivating glycogen synthase (12), is now recognized as a crucial intermediate in several intracellular signaling cascades. Specifically, GSK has been characterized as an intermediary of the Wnt signaling pathway, a role that has been shown to be key in regulating both in vitro and in vivo renewal of hematopoietic stem cells (13; 14). Previous studies demonstrate that GSK-3β modulates vascular progenitor cell function in vitro and in vivo resulting in enhanced EPC function, yield and ultimately in improved arterial repair following mechanical injury.

Current methods of treating patients with vascular disease, such as coronary heart disease or ischemic heart disease, have their inherent disadvantages. It is desirable to provide a safer and more effective device and method of promoting vascular repair, particularly in the treatment of vascular disease and other cardiovascular diseases.

### SUMMARY OF THE INVENTION

It is an object of the present invention to obviate or mitigate at least one disadvantage of previous devices and methods of treating vascular disease.

In a first aspect, the present invention provides a method of treating or preventing vascular disease in a subject, comprising administering to said subject a device having a kinase inhibitor.

In a further aspect of the present invention, there is provided a device for treating or preventing vascular disease in a subject, said device comprising a kinase inhibitor.

The device can be a stent, a balloon catheter, a local delivery catheter, or some other suitable means for local delivery. In one exemplary embodiment, the device is a stent.

The kinase inhibitor can be coated on the device or integrated therein using any methods known or contemplated in the art.

In a further aspect, the present invention provides a method of treating or preventing vascular disease in a subject, comprising administering to said subject a kinase inhibitor.

The kinase inhibitor can be a glycogen synthase kinase (GSK) inhibitor. In one embodiment, the GSK inhibitor is GSK-3β inhibitor.

Surprisingly, it has been found that the device and method of the present invention can be used to treat vascular disease such as coronary artery disease or ischemic heart disease, for example. Treatment can include promoting vascular repair. The device and method of the present invention can be used to treat cardiovascular disease in mammals, such as humans.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 illustrates the effects of GSK-3b inhibitor on the improvement of attenuated levels of EPCs from patients with CAD. FIGS. 1A and B show high and low power magnification of EPCs at 7 days labeled with DAPI (blue), AcLDL-Dil (Red), UEA-1-FITC (green), and merged image used for enumeration. FIG. 1C shows a Western blot of nuclear fraction from EPCs cultured in control (C) media and media supplemented with GSKI (2×) showing increased levels of b-catenin. FIG. 1D shows a comparison of EPC yields in both healthy controls and patients with CAD when treated with control (C) media, the 104 nm (1×) and 208 nm GSKI (2×), n=6. FIG. 1E shows that dual treatment of EPCs with both GSKI and LiCl does not synergistically improve EPC yields, n=6. * or # denote statistical significance, p<0.05.

FIG. 2 shows that GSKI enhances EPC survival and adherence. FIG. 2A shows that EPC survival is impaired in patients with CAD. Treatment with GSKI in both healthy controls and patients with CAD appears to significantly improves long term viability, n=6. FIG. 2B shows that GSKI decreases apoptosis in both EPCs derived from healthy controls and patients with CAD. FIG. 2C shows that GSKI improves adhesive properties of EPCs derived from both healthy controls and CAD patients, n=12. FIG. 2D shows that GSKI treatment upregulates mRNA of the alpha integrin isoform as measured by Q-PCR, n=6. FIG. 2E shows that introduction of a specific a4 integrin subunit blocking antibody demonstrates the reversibility of improved EPC adhesion and implicates a4 in EPC adhesion, n=6. * or # represent significant differences p<0.05.

FIG. 3 shows mouse femoral artery wire injury model of neointima formation. FIG. 3A shows wire injury results in significant increase in artery volume compared to sham. No significant differences exist between the vehicle group, the control EPCs (C-EPC) and the GSKI treated EPC (G-EPC) groups, n=6. FIG. 3B shows total intimal area and intima/media ratio in injured vessels injected with either normal saline, C-EPCs, and G-EPCs, n=6. FIG. 3C shows femoral arteries with hematoxylin and eosin staining. FIG. 3D shows intact and enface isolated femoral artery in Evans blue perfused mouse with de-endothelialized segment staining blue. FIG. 3E indicates the percentage of arterial re-endothelialization as assessed by Evans blue perfused arteries, n=5 for vehicle and n=6 for C-EPC and G-EPC groups. * or # denote statistical significance, p<0.05 or less.

FIG. 4 shows the effects of local GSKI delivery by drug coated stent at seven and fourteen days after stent implantation on neointima formation. FIG. 4A shows an image of vehicle gel coating on expanded stent. FIGS. 4B and C show representative arteries from vehicle coated (VCS) and GSKI coated stents (GCS). FIG. 4D show Day 7 and 14 neointimal area in bare metal stents (BMS), VCS, and GCSs. FIG. 4E shows Day 7 neointimal area comparing BMS, VCS, GCS, rapamycin coated stents (RCS), and the combination of GSKI and rapamycin coated stents (G+RCS). * represent significant differences p<0.05.

FIG. 5 shows the effects of local GSKI delivery by drug coated stent at seven and fourteen days after stent implantation on re-endothelialization. FIG. 5A shows GSKI coated stents (GCSs) have increased numbers of adherent EPCs compared to bare metal (BMS), vehicle coated (VCS), and rapamycin coated stents (RCS) in culture. Sample images of DAPI labeled EPCs on stent struts are inset. FIGS. 5B, C and D show representative scanning electron microscopy image of luminal surface of BMS, VCS, and GCSs respectively. FIG. 5E illustrates Day 7 and 14 re-endothelialization of BMS, VCS, and GCSs. FIG. 5F illustrates Day 7 re-endothelialization of BMS, VCS, GCS, rapamycin coated stents (RCS), and combination of GSKI and rapamycin coated statns (G+RCS). * or # indicates significant differences, p<0.05.

FIG. 6 shows GSK-3b inhibition with LiCl increases the yield of EPCs derived from both healthy controls (FIG. 6A) and CAD patients (FIG. 6B), n=6. * or # denote statistical significance, p<0.05 or less.

### DETAILED DESCRIPTION

Generally, the present invention provides a device and method for treating or preventing vascular disease.

In one aspect, the present invention provides a method of treating or preventing vascular disease in a subject, comprising administering to said subject a device having a kinase inhibitor. In another aspect of the present invention, there is provided a device for treating or preventing vascular disease in a subject, said device comprising a kinase inhibitor. The kinase inhibitor can be coated on the device or integrated therein using any methods known or contemplated in the art. The device can be a stent. In addition, the kinase inhibitor can be delivered locally using any other suitable device, such as a balloon or local delivery catheter.

In a further aspect, the present invention provides a method of treating or preventing vascular disease in a subject, comprising administering to said subject a kinase inhibitor.

The kinase inhibitor can be a glycogen synthase kinase (GSK) inhibitor. In one embodiment, the GSK inhibitor is GSK-3β inhibitor. The device and method can be used to treat vascular disease such as coronary artery disease or ischemic heart disease, for example. The device and method can be used to treat cardiovascular disease in mammals, such as humans. Typically, the device and method of the present invention can be used locally in arteries promote vascular repair.

The following abbreviations are used herein: CAD—coronary artery disease, DES—drug eluting stent, EGM-2—endothelial growth media-2, EPC—endothelial progenitor cell, GCS—GSKI coated stent, GSK—glycogen synthase kinase, GSKI—glycogen synthase kinase 3β inhibitor, LiCl—lithium chloride, UEA-1—ulex europeaus agluttinin-1, VLA—very late antigen.

### EXAMPLES

### Endothelial Progenitor Cell Culture

EPCs were isolated and cultured as previously described (17). Briefly, peripheral blood mononuclear cells (PBMCs) were isolated by ficoll gradient centrifugation from either healthy young volunteers or patients with angiographically verified CAD. Blood was collected by venipuncture and anticoagulated with EDTA. 5×10⁶ PBMCs were then washed and resuspended in EGM-2 media (Clonetics) and plated on fibronectin coated plates. Media were supplemented with either LiCl (Sigma), GSKI VIII (Calbiochem; Cat. No. 361549; AR-A014418:N-(4-Methoxybenzyl)-N′-(5-nitro-1,3-thiazol-2-yl)urea), or vehicle (DMSO) at the indicated concentrations.

GSK inhibitor VIII is a GSK-3β isoform specific inhibitor derived from a class of compounds shown to inhibit GSK-3 both in vitro (54) and in vivo (53). After 96 hours of culture, non-adherent cells were removed and the plates washed three times with buffered wash solution. Cells were then incubated for another 72 hours before being labeling, enumeration, or being used in various assays.

Cell Labeling and Western Blots

For the task of enumeration EPCs were arbitrarily defined as cells dually positive for AcLDL uptake and ulex europeus agglutinin I (UEAI) binding. Dil-AcLDL (2.5 μg/ml, Molecular Probes) was incubated with cultured EPCs for 1 hour in a cell incubator. Subsequently, cells were washed and fixed with Cytofix Buffer (BD) and incubated with FITC-UEAI (5 μg/ml, Sigma) for 30 minutes. Plates of cells were again washed, and incubated for a DAPI nuclear counterstain before a coverslip was applied to the well and double positive cells were counted in 6 random high power fields (×200 magnification).

β-catenin nuclear levels were assayed by western blot of nuclear extracts from both control and treated EPCs. Briefly, nuclei were separated by differential centrifugation and total nuclear protein was extracted. Protein was then run on an acrylamide gel and transferred onto a PVDF membrane overnight. After transfer, the membrane was blocked for one hour in 5% skim milk powder in 0.1% Tween-20 in Tris buffered saline. This was then incubated for 48 hours with primary antibody of a monoclonal anti-phospho-β-catenin (Cell Signaling Technologies) diluted 1:1000 in 5% skim milk powder in TBS-T. After incubation the membrane was then washed 3 times for 5 minutes each with TBS-T. The membrane was then incubated in secondary antibody of goat anti rabbit IgG (H+ L) conjugated with horseradish peroxidase (1:5000 in TBS-T) for 24 hours. The membrane was washed and quantitated using ECL Plus (Amersham Biosciences).

Cell Survival, Apoptosis, Migration, and VEGF Secretion Assays

Day seven EPCs were used for all experiments unless otherwise indicated. For the cell survival assay, six high power fields were enumerated on day 7 for each individual. Subsequently, cells were washed and the media changed every four days after which cells were again enumerated. Data are expressed as a percentage of initial cells present on day 7. For apoptosis studies cells from patients and healthy controls were cultured for 7 days. Cells were then lifted and recoated at a density of 2×10⁶ mature EPCs per well. The cells were then allowed to incubate for an additional four days prior to being lifted with EDTA supplementation of the media and gentle agitation. Cells were pelleted and resuspended in HBSS. Prior to analysis by flow cytometry, 10 μL of propidium iodide was added to the cell suspension. EPCs were identified by uptake of acLDL-alexa488 (Invitrogen) and UEA-1 FITC labeling in separate experiments. Uptake of propidium iodide identified apoptotic cells. A total of ten thousand events was analyzed and the data expressed as a percentage of total EPCs being apoptotic. All experiments were conducted on a Beckman Coulter Cytomics FC 500 cytometer.

Migration was performed with a Cytoselect 96-well fluorometric migration assay (Cell Biolabs) that uses a membrane with 8 μm pores. EPCs were lifted with 0.5 mM EDTA, pelleted, and resuspended in VEGF-free EGM-2. They were then counted on a haemocytometer and diluted to a concentration of 100,000 cells per 100 μl. The chemoattractant contained in the bottom chamber was one of either 150 μl of standard EGM-2 (VEGF supplemented) or VEGF-free EGM-2 supplemented with 100 ng/ml of stromal derived factor 1. Cells were allowed to migrate for 16 hours and then harvested with 150 μl per well of detachment solution. Cells were then incubated for 20 minutes and then shaken to remove all cells from the bottom of the membrane. Fifty μl of Cyquant/lysis buffer (1:75 in 4× lysis buffer) was added to each well of detached cells and then incubated for ten minutes. Next, 150 μl was removed to a new fluorometric capable 96 well plate (Corstar) and number of cells enumerated by excitation at 485 nm and emission of 520 nm. Relative fluorescence units were reported as a surrogate marker of number of migrated cells.

The secretion of VEGF by EPCs was measured using a VEGF ELISA kit (R&D Systems) using the manufacturer's provided protocol. Briefly, EPCs were plated in equal numbers and incubated in VEGF-free EGM-2 for 24 hrs. Subsequently, 200 μL of the culture supernatant was added to a 96 well plate coated with anti-human VEGF antibody. After 2 hours of incubation, the conjugated secondary antibody was added and allowed to incubate for another 2 hours. Substrate solution was added and the wells interrogated for absorption at 450 nm using a Bio-Rad micro-plate reader.

EPC Adhesion

Day 7 EPCs were detached from their fibronectin coated plates by incubation with 0.5 mmol EDTA. Cells were pelleted, resuspended in EGM-2 and enumerated. Subsequently, 100,000 EPCs were plated in 24-well fibronectin coated plates and incubated for 30 minutes. After, the wells were washed three times with HBSS and the adherent cells labeled and six random high power fields enumerated. Blocking experiments were performed using a specific VLA-4 antibody (MAB169832, Chemicon) at a concentration of 10 ug/mL and allowing cells to incubate for 2 hours.

Quantitative PCR was performed on the Light Cycler Q-PCR System (Roche) and data analyzed using the accompanying software package. Total RNA was isolated from EPCs using Trizol (Invitrogen) and RT performed using standard techniques. Amplicons were cloned into the pGEM-T vector, sequenced, and isolated using the PhasePrep BAC DNA kit (Sigma). The plasmids were then linearized, purified, and diluted to generate standard curves for quantitative PCR analysis.

Primers were designed using the PrimerQuest software and were as follows:

| | |
|---|---|
| GAPDHfwd: | CGCCTGGAGAAAGCTGCTAAGTAT, |
| GAPDHrev: | GCTTCACAAAGTGGTCATTGAGGG, |
| VLA1fwd: | ACAAGTGACAGCGAAGAACCTCCT, |
| VLA1rev: | TGGGTACAGCACAGGGTAACCATT, |
| VLA2fwd: | ACTTTATCTCCAGCGGTACAAAGT, |
| VLA2rev: | TGGGCCTTATCCCAATCTGACCAA, |
| VLA3fwd: | CAAAGACAGGCAAACGGCAACGTA, |
| VLA3rev: | TTATTGGTCGCGGTGAGAAGCCTA, |
| VLA4fwd: | AGGGCAAGGAAGTTCCAGGTTACA, |
| VLA4rev: | ACATGAGGACCAAGGTGGTAAGCA, |
| VLA5fwd: | TGCCTGAGTCCTCCCAATTTCAGA, |
| VLA5rev: | ACATGAGGACCAAGGTGGTAAGCA. |

PCR was performed with an annealing temperature of 56 degrees for all primer combinations. The QPCR reagents utilized were the QuantiTect SYBR PCR system and the QuantiTect Probe PCR system (Qiagen). All primer combinations were confirmed to have a single amplicon on agarose gel and SYBR green PCR was utilized for quantification of alpha integrins 1-5 and GAPDH. Confirmation of VLA4 integrin mRNA upregulation was done using a probe specific quantitative PCR technique. The VLA4fwd and VLA4rev primers were used in conjunction with a 5′ 6-FAM labeled and 3′ TAMRA modified probe VLA4probe: AGCATTTATGCGGAAAGATGTGCGGG. For these experiments, Qiagen QuantiTect Probe PCR system was utilized.

CD-1 Nude Mouse Femoral Artery Wire Injury Model

CD-1 nude mice were acquired from Charles River Laboratories and acclimatized in our facilities for 2-6 weeks prior to surgeries. Femoral artery injury was induced by insertion of a 32 gauge blunt needle (Strategic Applications Inc) to induce neointima formation as previously described (26). Briefly, CD-1 nude mice were anesthetized, prepped and the femoral artery dissected unilaterally. Distal to major branches, as to ensure continued flow threw the injured segment, two sutures were passed under the femoral artery and the vessel lifted to interrupt blood flow. The artery was then incised and the syringe introduced into the lumen, advanced proximally and passed five times to denude endothelium and mechanically stretch the vessel. Subsequently, 5×10⁵ of control EPCs, GSKI treated EPCs or vehicle (n=6) were injected and the syringe removed keeping tension on the proximal suture to ensure retention of administered cells. Both sutures were then firmly tied to prevent exsanguination. As the incision and sutures were distal to major femoral artery branch points flow continued in the injured segment and none of the limbs became ischemic.

Rabbit Carotid Stenting Model

GCSs were generated using techniques previously described (27). Briefly, to generate the GSK coating gel, GSKI (25 mM in DMSO from Calbiochem was reconstituted in water to make concentration at 20×IC₅₀) was mixed with Lubricating Jelly (Medline Industries) at ratio of 1:9. The vehicle control coating gel was made by mixing 0.01% DMSO in water with Lubricating Jelly at ratio of 1:9. Twelve stents (Driver 3.0×24 mm, Medtronic) were manually coated with either 45 μl (45 μg) GSK3I coating gel or vehicle coating gel (n=6).

Six male New Zealand white rabbits (3.0-3.5 kg, Charles River Laboratories, Quebec, Canada) were studied. Under general anesthesia with ketamine [25 mg/kg, intramuscularly (i.m.)], midazolam (2-4 mg/kg, i.m.), and isoflurane (via an endrotracheal tube), a GCSs or vehicle coated stent was deployed at six atmospheres in each carotid artery. All rabbits received two stents (one on each side) with both stents having either GSKI or vehicle coating. All animals received heparin (125 U/kg, Leo Pharma) as an intravenous bolus at the outset of the procedure. To limit stent thrombosis, all rabbits were given acetylsalicylic acid (rectal gel, 10 mg/kg, per os) every day, starting 3 days before stenting and continuing until euthanization. As per usual clinical practice, the complementary antiplatelet agent clopidogrel bisulfate (Sanofi-Synthelabo) was administered transdermally, beginning with a loading dose of 4 mg/kg on the day before stenting and continued as 1 mg/kg/day thereafter until euthanasia.

Tissue Harvest and Quantitative Histomorphologic Analyses

Fourteen days after stent implantation or femoral artery injury, animals were euthanized and the stented carotid arteries or injured femoral arteries were harvested. Femoral arteries were fixed in buffered formalin then dehydrated with ethanol. Arteries were mounted in paraffin blocks and sectioned in 5 μm sections at 250 and 500 μm from the proximal suture to standardize the region quantified between samples. Sections were HE-stained and analysis performed using a computer-assisted digital imaging system (Image-Pro Plus, Media Cybernetics).

Stented arteries were divided into 4 segments evenly as following. The first segment was embedded in methylmethacrylate after overnight fixation with 10% neutral-buffered formalin (NBF). Cross-sections (5 μm-thick) were cut with a D-Profile tungsten carbide knife (Delaware Diamond Knives), and hematoxylin/eosin stained slides were obtained. The lumen area (LA) as well as the area circumscribed by the internal elastic lamina (IEL) was measured on HE-stained sections using the computer-assisted digital imaging system. Neointimal (NI) area was defined as: NI area=IEL area minus the LA. The second segment was dissected open and the stent was manually removed before fixation in 10% NBF. Tissue samples were then embedded in paraffin afterwards. The third segment was similarly opened, the stent manually removed, and the tissue snap frozen and embedded in optimal cutting temperature compound. For both paraffin and frozen tissue blocks, serial 5 μm cross-sections were cut at subsegment intervals of 350 μm. For morphometric analyses, nine cross-sections from three subsegments (three cross-sections per subsegment) were examined. The fourth segment was utilized for scanning electron microscopy (SEM) and was dissected open longitudinally, flattened, and fixed in 1.6% glutaraldehyde before being dehydrated and dried with liquid CO₂. The samples were coated with gold and examined using SEM (XL 30 ESEM, Philips Electronics). SEM photomicrographs of each specimen were specifically examined for reconstitution of the endothelium. For each specimen, SEM photomicrographs were taken at 5 spots at 400× magnifications. Percentages of re-endothelialization area vs total surface area were analyzed using the computer-assisted digital system.

Tissue Samples and Statistics

Animal procedures were performed with the approval of the University of Ottawa Animal Care Committee and followed the guidelines of the Canadian Council on Animal Care. All protocols involving human donors were approved by the Ottawa Heart Institute Research Ethics Board.

For all statistical procedures a p-value less than 0.05 was considered statistically significant. All analysis was performed using the Sigmastat and Systat statistical packages (SYSTAT). Two way comparisons between groups was performed with a student's t-test. Multiple group comparisons were performed using one-way ANOVAs with Holm-Sidak post-hoc tests for pairwise comparisons. Data are expressed as means+/−standard error of the mean.

Results

Revascularization of the coronary circulation by percutaneous intervention has typically been a preferred strategy in patients with ischemic heart disease. Use of drug eluting stents reduce neointimal growth and revascularization rates but with a clinically relevant delay in re-endothelialization (RE). Previous work has demonstrated enhanced endothelial progenitor cell (EPC) numbers and function by treatment with a glycogen synthase kinase 3-beta inhibitor (GSKI).

Following wire injury vehicle, transplantation of human EPCs or GSKI-treated EPCs showed a significant increase in RE assessed by en-face Evans blue stained arteries (7.2±1.7% vs. 70.7±5.8% vs. 87.2±4.1%, p<0.05). In an in vitro study of EPC culture in the presence of vehicle (VCS), GSKI (GCS), and rapamycin-coated stents (RCS), GSKI coating resulted in a 20-fold increase in EPC attachment when compared to rapamycin coated stents and a 2-fold increase compared to control (21.5±2.7 vs 1.5±0.8 vs 10.2±1.6, p<0.05).

The beneficial effect of GCSs was demonstrated in a New Zealand White rabbit carotid stenting model. Bare metal stents (BMSs), VCSs, GCSs, or RCSs were examined for RE at 7 days. Scanning electron microscopy showed a marked increase in RE of GCSs compared to VCSs and BMSs respectively (65.5±6.4% vs 46.7±3.8% vs 49.4±3.2%, p<0.05). Similarly, dually coating stents with both GSKI and rapamycin improved RE compared to baseline (63.0±4.5%, p<0.01). At 14 days, GCSs also demonstrated reductions in neointima formation by 50% relative to VCSs (p<0.05).

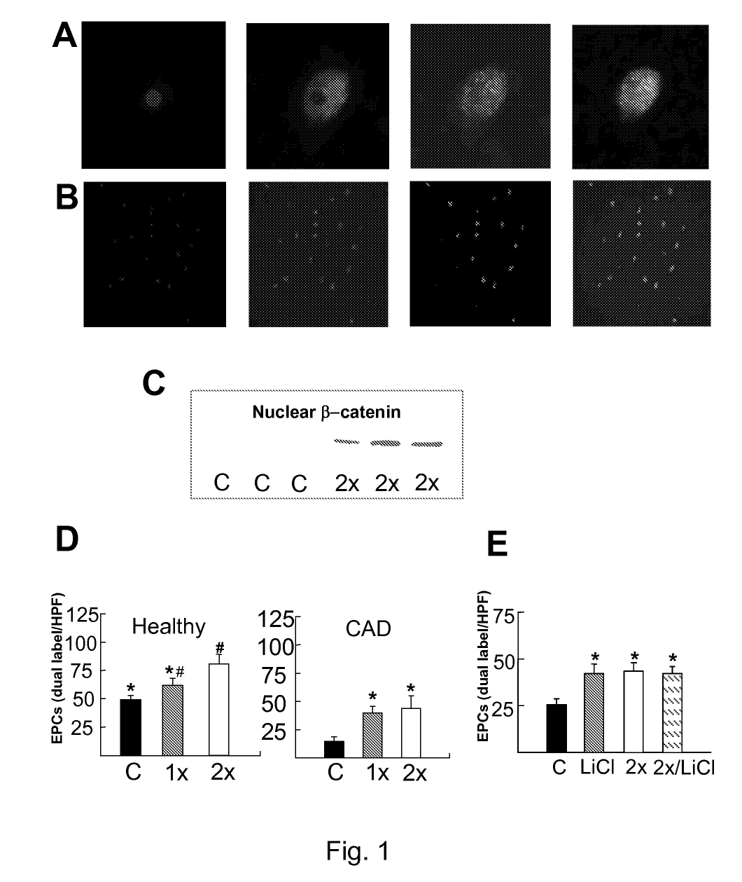

FIG. 1 illustrates the effects of GSK-3b inhibitor on the improvement of attenuated levels of EPCs from patients with CAD. Peripheral blood mononuclear cells (PBMCs; 5×10⁶) isolated from both healthy patients and controls were cultured for 7 days in endothelial growth media-2 (EGM-2) on fibronectin coated dishes and cells showing ac-LDL uptake and ulex europeaus agluttinin-1 (UEA-1) dual labeling cells were enumerated. Compared to healthy controls, patients with CAD had 3 times fewer EPCs (48.5+/−4.6 vs. 14.0+/−3.2; p<0.05). EGM-2 media was then supplemented with GSK-3β inhibitor VIII (Calbiochem) at 1× and 2× the IC50 dose (104 nm and 208 nm; respectively). Supplementing media with the inhibitor at both concentrations lead to an increase in EPC yield in both healthy controls and CAD patients.

FIGS. 1A and B show high and low power magnification of EPCs at 7 days labeled with DAPI (blue), AcLDL-Dil (Red), UEA-1-FITC (green), and merged image used for enumeration. FIG. 1C shows a Western blot of nuclear fraction from EPCs cultured in control (C) media and media supplemented with GSKI (2×) showing increased levels of b-catenin. FIG. 1D shows a comparison of EPC yields in both healthy controls and patients with CAD when treated with control (C) media, the 104 nm (1×) and 208 nm GSKI (2×), n=6. FIG. 1E shows that dual treatment of EPCs with both GSKI and LiCl does not synergistically improve EPC yields, n=6. * or # denote statistical significance, p<0.05.

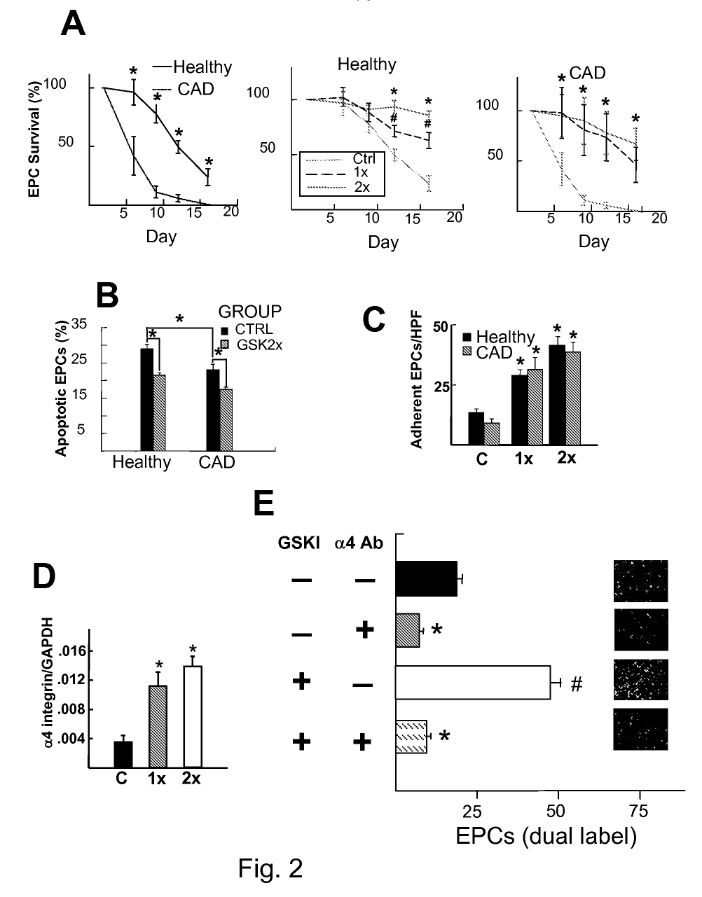

FIG. 2 shows that GSKI enhances EPC survival and adherence. Experimental evidence from clinical studies suggest that EPC functional capacity may be equally as important as absolute numbers of progenitor cells for in vivo activity (19). The ability of EPCs to home to sites of injury, migrate, and secrete cytokines is integral for effective arterial repair. Given the evidence that GSK-3β signaling is important in regulation of apoptosis (20), it has been thought that the improved survival observed may be linked to lower levels of apoptosis. To test this hypothesis we cultured EPCs for 7 days then replated them at equal densities and treated them with either control EGM-2 media or 2×GSKI. Using propidium iodide (21), the percentage of apoptotic EPCs in both healthy controls and patients was analyzed. FIG. 2A shows that EPC survival is impaired in patients with CAD. Treatment with GSKI in both healthy controls and patients with CAD appears to significantly improves long term viability, n=6. FIG. 2B shows that GSKI decreases apoptosis in both EPCs derived from healthy controls and patients with CAD. FIG. 2C shows that GSKI improves adhesive properties of EPCs derived from both healthy controls and CAD patients, n=12. FIG. 2D shows that GSKI treatment upregulates mRNA of the alpha integrin isoform as measured by Q-PCR, n=6. FIG. 2E shows that introduction of a specific a4 integrin subunit blocking antibody demonstrates the reversibility of improved EPC adhesion and implicates a4 in EPC adhesion, n=6. * or # represent significant differences p<0.05. At baseline, CAD-EPCs demonstrated a higher rate of apoptosis when compared to healthy controls. In both groups, 2×GSKI resulted in a reduction in the percentage of apoptotic cells observed—a result which may explain the improved survival observed in GSKI treated cells. Notably, there was no difference between treated CAD-EPCs and treated healthy control cells with regard to the frequency of apoptosis when treated with GSKI. These data suggest that inhibition of GSK-3β attenuates the apoptosis of EPCs from patients with CAD to levels equal to those of healthy controls likely resulting in enhanced EPC survival.

The ability of GSKI to affect relevant cytokine secretion and EPC migration was tested. Equal numbers of mature EPCs were incubated for 24 hours in VEGF free media before VEGF levels were measured by ELISA. As previously demonstrated in EPCs transfected with a GSK-3β dominant negative mutant (16), an increase in VEGF secretion by mature EPCs was observed. These data appear to demonstrate that GSKI not only results in increased yields of EPCs, but also enhances some characteristics of these progenitors potentially optimizing them for therapeutic use.

A key step in EPC incorporation in a vessel wall involves adhesion at the target site (22). Clinically, the adhesiveness of EPCs has been implicated in arterial repair following stent deployment (23). The adherence of EPCs in a fibronectin adhesion assay comparing cells of healthy controls vs. patients with CAD was tested, and no difference was found. Treatment of both populations with GSKI resulted in an approximate 4 fold increase in patients cells with a dose dependent increase in healthy controls (p<0.05).

Previous studies indicate that the integrin family of cell surface receptors play a key role in adhesion and integration of circulating progenitor cells into the vasculature (24; 25). The mRNA abundance of these α-integrin subunits with GSKI treatment was determined. The α-integrin subunits 1-5 were cloned as well as the GAPDH gene from human PBMCs and SYBR green quantitative PCR was performed on mRNA from control, GSKI 1×, and GSKI 2× treated EPC samples. No difference was found in mRNA levels of α-1, -2, -3 and -5 integrin subunits between control and treatment samples. In contrast, the α-4 integrin subunit showed upregulation with both concentrations of the GSKI. These findings were then confirmed with a probe specific Q-PCR reaction to ensure the specificity of the results.

A VLA4 blocking antibody was used in the fibronectin adhesion assay to determine if upregulation of the α-4 integrin subunit is linked to the observed enhancement of EPC adhesion. Addition of GSKI resulted in an increased number of adherent cells after 30 minutes. Notably, this effect was completely abrogated by pretreatment of the cells with the VLA4 blocking antibody—thereby confirming that the observed increase in adhesion is specifically mediated through the α-4 integrin subunit.

Werner et al. (7) first described the ability of ex vivo cultured EPCs to mediate arterial repair in vivo. In accordance with one aspect of the present invention, improved in vivo function achieved by GSK inhibition was determined. Human EPCs were treated ex vivo under normal conditions and in media supplemented with GSKI before systemically infusing these cells into immune compromised mice subjected to femoral artery wire injury. In particular, the effects of GSK inhibition on cells from patients with CAD who at baseline have fewer cells with impaired qualitative properties, were sought. Cells from six patients with CAD were cultured as previously described or in EGM-2 media supplemented with GSKI. On day 7, EPCs were resuspended in sterile normal saline. The femoral arteries of CD1 athymic nude mice were dissected, isolated, incised and a 32 gauge blunt syringe introduced into the lumen of these vessels. The syringe was passed to and fro in the artery to ensure endothelial denudation and mechanical injury to the artery (26). Subsequently, the ex vivo treated cells were injected intra-arterially. Notably, the femoral artery was incised and subsequently ligated distal to major branches to ensure that flow through the artery was not eliminated and that the limb did not become ischemic. Fourteen days after injury the mice were sacrificed and the arteries assessed for neointimal formation.

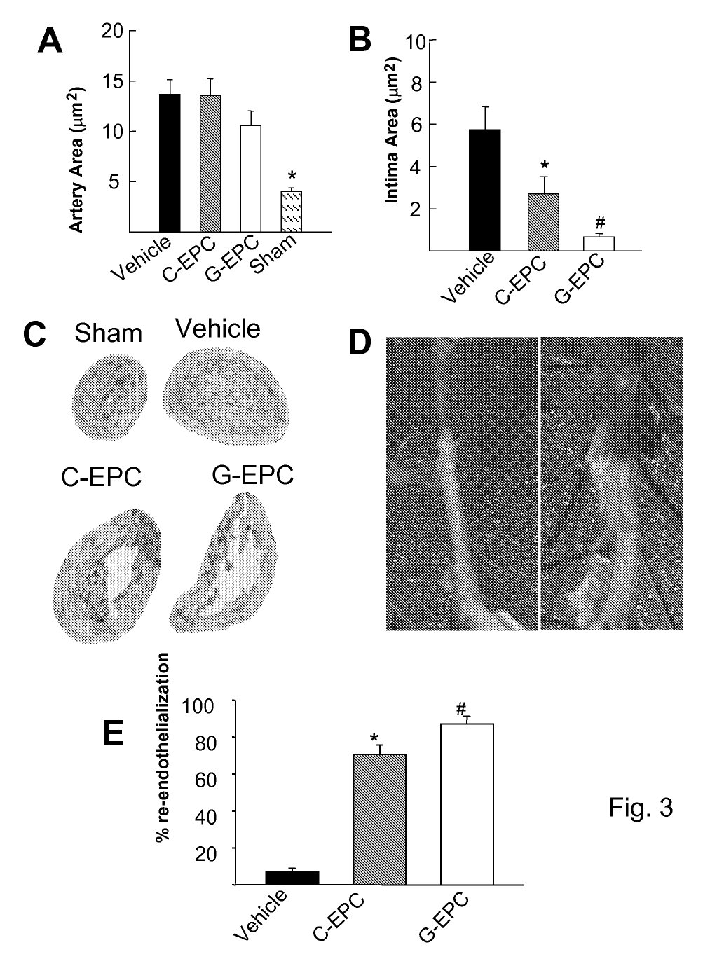

FIG. 3 shows mouse femoral artery wire injury model of neointima formation. FIG. 3A shows wire injury results in significant increase in artery volume compared to sham. No significant differences exist between the vehicle group, the control EPCs (C-EPC) and the GSKI treated EPC (G-EPC) groups, n=6. FIG. 3B shows total intimal area and intima/media ratioin injured vessels injected with either normal saline, C-EPCs, and G-EPCs, n=6. FIG. 3C shows femoral arteries with hematoxylin and eosin staining. FIG. 3D shows intact and enface isolated femoral artery in Evans blue perfused mouse with de-endothelialized segment staining blue. FIG. 3E indicates the percentage of arterial re-endothelialization as assessed by Evans blue perfused arteries, n=5 for vehicle and n=6 for C-EPC and G-EPC groups. * or # denote statistical significance, p<0.05 or less.

When compared to sham (uninjured) arteries the injured arteries were nearly 3 times larger reflecting marked mechanical dilatation of the lumen. Notably, there were no differences among the treatment groups, possibly suggesting that the degree of injury did not vary. Neointima formation was quantified and averaged using hematoxylin and eosin stained tissue cross sections obtained 250 and 500 μm proximal to the ligation site. Both total intimal area and the ratio of neointimal to medial area were calculated. EPC infusion alone resulted in a reduction in neointima volume as previously reported (7). Culture of EPCs with GSKI resulted in a marked further reduction in neointimal formation even compared to control EPCs. Notably, there were no differences in medial area when comparing between the various treatment groups. These studies confirm that enhanced EPC function in vitro translated into superior reparative capacity of transplanted cells in vivo.

In accordance with another aspect of the present invention, local delivery of inhibitor at the site of arterial injury represents an alternative strategy for enhancing arterial repair and mitigating neointima formation. GSKI coated stents (GCSs) were manufactured for use in a rabbit carotid model of stent neointima formation as previously described (27). Prior to implanting these GCSs, in vitro experiments were performed in order to predict the maximal tolerated dose of GSKI we could deliver in vivo. EPCs were cultured in EGM-2 supplemented with 1×, 2×, 5×, 10×, 20×, 30×, 40×, 50×, and 100× concentrations of the GSKI. The 30× and higher doses resulted in increased EPC death and attenuated function; thus, the 20× dose was used for generating the GCSs. To manufacture the GCSs, bare metal stents were coated with muco-gel supplemented with DMSO (vehicle for GSKI) or muco-gel containing 20×GSKI as previously described (27). Once coated, stents were allowed to dry in a sterile environment and packaged aseptically prior to delivery. Stents were deployed in New Zealand white rabbit carotid arteries according to a previous protocol and the stent neointima was subjected to quantitative histomophometry 14 days post-stent deployment (22). Scanning electron microscopy on frozen sections to quantify stent strut re-endothelialization and noted near complete coverage in the GCS group with comparatively sparse single cells on control stents. Indeed, stent struts from GCSs showed a 30% increase in re-endothelialization when compared to controls. Despite rapid re-endothelialization of GCSs, a neointima formed in both groups but was 50% smaller in area in GCSs vs. control stents. These studies indicate that local delivery of a GSK inhibitor is also an effective method of improving arterial healing via promotion of re-endothelialization.

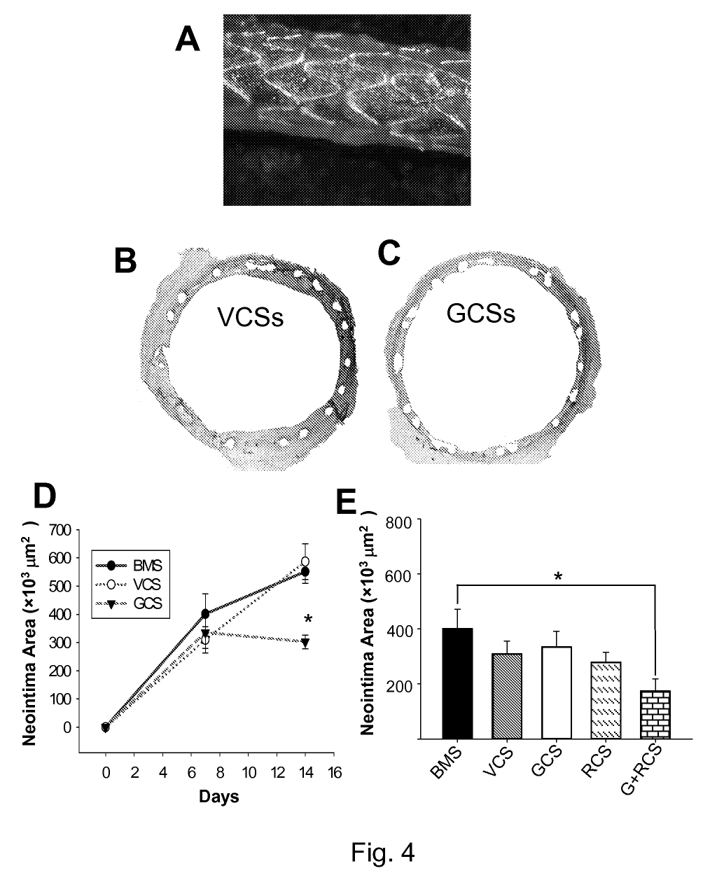

FIG. 4 shows the effects of local GSKI delivery by drug coated stent at seven and fourteen days after stent implantation on neointima formation. FIG. 4A shows an image of vehicle gel coating on expanded stent. FIGS. 4B and C show representative arteries from vehicle coated (VCS) and GSKI coated stents (GCS). FIG. 4D show Day 7 and 14 neointimal area in bare metal stents (BMS), VCS, and GCSs. FIG. 4E shows Day 7 neointimal area comparing BMS, VCS, GCS, rapamycin coated stents (RCS), and the combination of GSKI and rapamycin coated stents (G+RCS). * represent significant differences p<0.05.

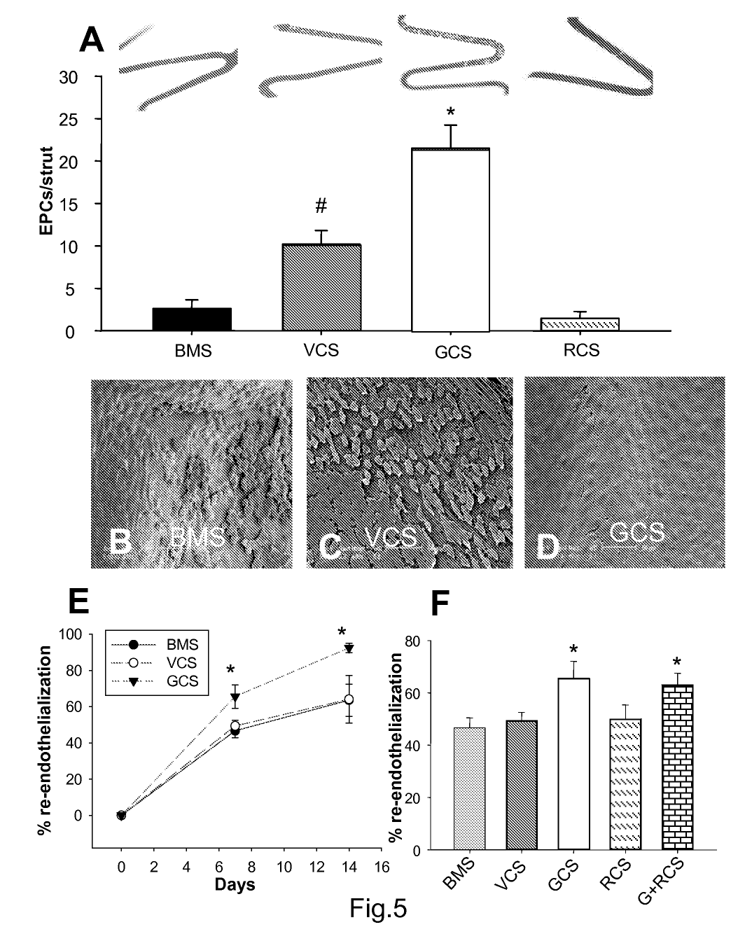

FIG. 5 shows the effects of local GSKI delivery by drug coated stent at seven and fourteen days after stent implantation on re-endothelialization. FIG. 5A shows GSKI coated stents (GCSs) have increased numbers of adherent EPCs compared to bare metal (BMS), vehicle coated (VCS), and rapamycin coated stents (RCS) in culture. Sample images of DAPI labeled EPCs on stent struts are inset. FIGS. 5B, C and D show representative scanning electron microscopy image of luminal surface of BMS, VCS, and GCSs respectively. FIG. 5E illustrates Day 7 and 14 re-endothelialization of BMS, VCS, and GCSs. FIG. 5F illustrates Day 7 re-endothelialization of BMS, VCS, GCS, rapamycin coated stents (RCS), and combination of GSKI and rapamycin coated statns (G+RCS). * or # indicates significant differences, p<0.05.

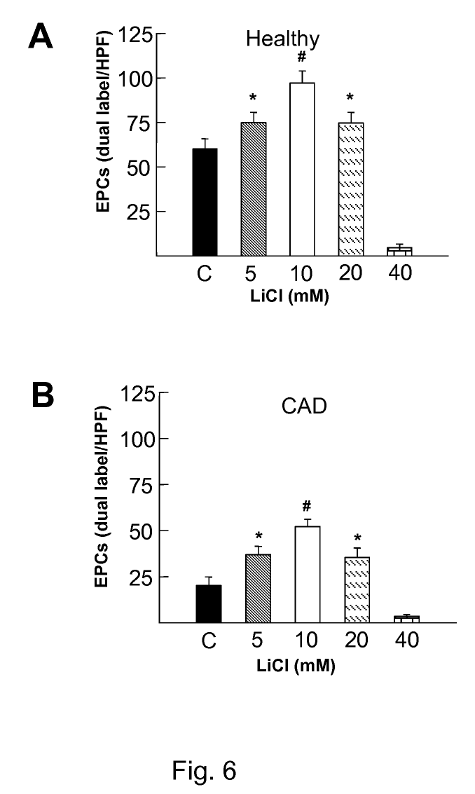

EPCs were cultured with varying doses of LiCl—as LiCl inhibits GSK-3β in doses ranging from 10-20 mmolar (18). FIG. 6 shows GSK-3b inhibition with LiCl increases the yield of EPCs derived from both healthy controls (FIG. 6A) and CAD patients (FIG. 6B), n=6. * or # denote statistical significance, p<0.05 or less. Combination treatment with GSKI and LiCl failed to result in any further improvement in EPC yields suggesting that these two methods of treatment improved EPC yields via a specific action on the GSK-3β isoform.

The above indicates that inhibition of GSK-3 appears to improve RE by EPCs and that local delivery by GSKI coated stent may augment EPC recruitment and RE resulting in reductions in neointima formation following arterial stenting.

The present invention shows that GSKI enhances not only EPC number and survival, but also adhesion via upregulation of the α4 integrin subunit. Further, a 75% reduction in neointimal formation with transplantation of EPCs from patients with CAD that were cultured in media supplemented with GSKI compared to normal conditions. Finally, using a novel vascular stent coated with a GSKI-containing gel we observed not only a reduction in stent neointimal area but also a remarkable increase in endothelial regeneration within the stented segment.

The present invention provides a comparable 50% reduction in neointimal area with the addition of complete re-endothelialization. These findings suggest that local delivery of agents designed to inhibit GSK-3β may provide an alternative strategy for reducing neointimal formation while also promoting quick re-endothelialization—a result of great clinical significance. Progenitor cells may also be modified ex vivo by administering to said cells a kinase inhibitor. The number of progenitor cells may also be modified by administering to said cells a kinase inhibitor. The progenitor cells may be modified in vivo, in vitro, ex vivo followed by administration in vivo, or any other method known or contemplated in the art.

Inhibition of GSK-3β enhances the yield of EPCs in vitro and promotes EPC-mediated arterial healing in vivo by both cell based and local delivery strategies. Thus, in accordance with one aspect of the present invention, the enhancement of arterial healing following mechanical injury can be achieved.

The above-described embodiments of the present invention are intended to be examples only. Alterations, modifications and variations may be effected to the particular embodiments by those of skill in the art without departing from the scope of the invention, which is defined solely by the claims appended hereto.

### REFERENCE LIST

1. Froeschl, M., Olsen, S., Ma, X., and O'Brien, E. R. 2004. Current understanding of in-stent restenosis and the potential benefit of drug eluting stents. Current Drug Targets—Cardiovascular & Haematological Disorders 4:103-117.
2. Serruys, P. W., Kutryk, M. J., and Ong, A. T. 2006. Coronary-artery stents. N. Engl. J Med. 354:483-495.
3. Luscher, T. F., Steffel, J., Eberli, F. R., Joner, M., Nakazawa, G., Tanner, F. C., and Virmani, R. 2007. Drug-eluting stent and coronary thrombosis: biological mechanisms and clinical implications. Circulation 115:1051-1058.
4. Strehlow, K., Werner, N., Berweiler, J., Link, A., Dirnagl, U., Priller, J., Laufs, K., Ghaeni, L., Milosevic, M., Bohm, M. et al 2003. Estrogen increases bone marrow-derived endothelial progenitor cell production and diminishes neointima formation. Circulation 107:3059-3065.
5. Walter, D. H., Rittig, K., Bahlmann, F. H., Kirchmair, R., Silver, M., Murayama, T., Nishimura, H., Losordo, D. W., Asahara, T., and Isner, J. M. 2002. Statin therapy accelerates reendothelialization: a novel effect involving mobilization and incorporation of bone marrow-derived endothelial progenitor cells. Circulation 105:3017-3024.
6. Kong, D., Melo, L. G., Gnecchi, M., Zhang, L., Mostoslavsky, G., Liew, C. C., Pratt, R. E., and Dzau, V. J. 2004. Cytokine-induced mobilization of circulating endothelial progenitor cells enhances repair of injured arteries. Circulation 110:2039-2046.
7. Werner, N., Junk, S., Laufs, U., Link, A., Walenta, K., Bohm, M., and Nickenig, G. 2003. Intravenous transfusion of endothelial progenitor cells reduces neointima formation after vascular injury. Circ Res 93:e17-e24.
8. Assmus, B., Honold, J., Schachinger, V., Britten, M. B., Fischer-Rasokat, U., Lehmann, R., Teupe, C., Pistorius, K., Martin, H., Abolmaali, N. D. et al 2006. Transcoronary transplantation of progenitor cells after myocardial infarction. N. Engl. J Med. 355:1222-1232.
9. Schachinger, V., Erbs, S., Elsasser, A., Haberbosch, W., Hambrecht, R., Holschermann, H., Yu, J., Corti, R., Mathey, D. G., Hamm, C. W. et al 2006. Intracoronary bone marrow-derived progenitor cells in acute myocardial infarction. N. Engl. J Med. 355:1210-1221.
10. Hill, J. M., Zalos, G., Halcox, J. P., Schenke, W. H., Waclawiw, M. A., Quyyumi, A. A., and Finkel, T. 2003. Circulating endothelial progenitor cells, vascular function, and cardiovascular risk. N. Engl. J Med. 348:593-600.
11. Heeschen, C., Lehmann, R., Honold, J., Assmus, B., Aicher, A., Walter, D. H., Martin, H., Zeiher, A. M., and Dimmeler, S. 2004. Profoundly reduced neovascularization capacity of bone marrow mononuclear cells derived from patients with chronic ischemic heart disease. Circulation 109:1615-1622.
12. Cohen, P., Nimmo, H. G., and Proud, C. G. 1978. How does insulin stimulate glycogen synthesis? Biochem. Soc. Symp. 69-95.
13. Reya, T., Duncan, A. W., Ailles, L., Domen, J., Scherer, D. C., Willert, K., Hintz, L., Nusse, R., and Weissman, I. L. 2003. A role for Wnt signalling in self-renewal of haematopoietic stem cells. Nature 423:409-414.
14. Trowbridge, J. J., Xenocostas, A., Moon, R. T., and Bhatia, M. 2006. Glycogen synthase kinase-3 is an in vivo regulator of hematopoietic stem cell repopulation. Nat. Med. 12:89-98.
15. Wang, H. Y., Gao, P. J., Ji, K. D., Shen, W. F., Fan, C. L., Lu, L., and Zhu, D. L. 2007. Circulating endothelial progenitor cells, C-reactive protein and severity of coronary stenosis in Chinese patients with coronary artery disease. Hypertens. Res. 30:133-141.
16. Choi, J. H., Hur, J., Yoon, C. H., Kim, J. H., Lee, C. S., Youn, S. W., Oh, I. Y., Skurk, C., Murohara, T., Park, Y. B. et al 2004. Augmentation of therapeutic angiogenesis using genetically modified human endothelial progenitor cells with altered glycogen synthase kinase-3beta activity. J Biol. Chem. 279:49430-49438.
17. Hibbert, B., Chen, Y. X., and O'Brien, E. R. 2004. c-kit-immunopositive vascular progenitor cells populate human coronary in-stent restenosis but not primary atherosclerotic lesions. Am. J. Physiol Heart Circ. Physiol 287:H518-H524.
18. Klein, P. S., and Melton, D. A. 1996. A molecular mechanism for the effect of lithium on development. Proc. Natl. Acad. Sci. U.S. A 93:8455-8459.
19. Seeger, F. H., Tonn, T., Krzossok, N., Zeiher, A. M., and Dimmeler, S. 2007. Cell isolation procedures matter: a comparison of different isolation protocols of bone marrow mononuclear cells used for cell therapy in patients with acute myocardial infarction. Eur. Heart J 28:766-772.
20. Pap, M., and Cooper, G. M. 1998. Role of glycogen synthase kinase-3 in the phosphatidylinositol 3-Kinase/Akt cell survival pathway. J Biol. Chem. 273:19929-19932.
21. Riccardi, C., and Nicoletti, I. 2006. Analysis of apoptosis by propidium iodide staining and flow cytometry. Nat Protoc. 1:1458-1461.
22. Ma, X., and O'Brien, E. R. 2004. Antagonism of the alpha-4 integrin subunit attenuates the acute inflammatory response to stent implantation yet is insufficient to prevent late intimal formation. J Leukocyte Biol 75:1016-1021.
23. George, J., Herz, I., Goldstein, E., Abashidze, S., Deutch, V., Finkelstein, A., Michowitz, Y., Miller, H., and Keren, G. 2003. Number and Adhesive Properties of Circulating Endothelial Progenitor Cells in Patients With In-Stent Restenosis. Arterioscler. Thromb. Vasc. Biol. 23:57e-60.
24. Jin, H., Aiyer, A., Su, J., Borgstrom, P., Stupack, D., Friedlander, M., and Varner, J. 2006. A homing mechanism for bone marrow-derived progenitor cell recruitment to the neovasculature. J Clin. Invest 116:652-662.
25. Peled, A., Grabovsky, V., Habler, L., Sandbank, J., renzana-Seisdedos, F., Petit, I., Ben-Hur, H., Lapidot, T., and Alon, R. 1999. The chemokine SDF-1 stimulates integrin-mediated arrest of CD34(+) cells on vascular endothelium under shear flow. J Clin. Invest 104:1199-1211.
26. Sata, M., Maejima, Y., Adachi, F., Fukino, K., Saiura, A., Sugiura, S., Aoyagi, T., Imai, Y., Kurihara, H., Kimura, K. et al 2000. A mouse model of vascular injury that induces rapid onset of medial cell apoptosis followed by reproducible neointimal hyperplasia. J. Mol Cell Cardiol. 32:2097-2104.
27. Chen, Y. X., Ma, X., Whitman, S., and O'Brien, E. R. 2004. Novel antiinflammatory vascular benefits of systemic and stent-based delivery of ethylisopropylamiloride. Circulation 110:3721-3726.
28. Hill, J. M., Syed, M. A., Arai, A. E., Powell, T. M., Paul, J. D., Zalos, G., Read, E. J., Khuu, H. M., Leitman, S. F., Horne, M. et al 2005. Outcomes and risks of granulocyte colony-stimulating factor in patients with coronary artery disease. J Am. Coll. Cardiol 46:1643-1648.
29. Seeger, F. H., Zeiher, A. M., and Dimmeler, S. 2007. Cell-enhancement strategies for the treatment of ischemic heart disease. Nat. Clin. Pract. Cardiovasc. Med. 4 Suppl 1:S110-S 113.
30. Yost, C., Torres, M., Miller, J. R., Huang, E., Kimelman, D., and Moon, R. T. 1996. The axis-inducing activity, stability, and subcellular distribution of beta-catenin is regulated in Xenopus embryos by glycogen synthase kinase 3. Genes Dev. 10:1443-1454.
31. Koyanagi, M., Haendeler, J., Badorff, C., Brandes, R. P., Hoffmann, J., Pandur, P., Zeiher, A. M., Kuhl, M., and Dimmeler, S. 2005. Non-canonical Wnt signaling enhances differentiation of human circulating progenitor cells to cardiomyogenic cells. J Biol. Chem. 280:16838-16842.
32. Bakre, M. M., Hoi, A., Mong, J. C., Koh, Y. Y., Wong, K. Y., and Stanton, L. W. 2007. Generation of Multipotential Mesendodermal Progenitors from Mouse Embryonic Stem Cells via Sustained Wnt Pathway Activation. J Biol. Chem. 282:31703-31712.
33. Fattori, R., and Piva, T. 2003. Drug-eluting stents in vascular intervention. Lancet 361:247-249.
34. Hillebrands, J. L., Klatter, F. A., and Rozing, J. 2003. Origin of vascular smooth muscle cells and the role of circulating stem cells in transplant arteriosclerosis. Arterioscler. Thromb. Vasc. Biol. 23:380-387.
35. Tanaka, K., Sata, M., Hirata, Y., and Nagai, R. 2003. Diverse contribution of bone marrow cells to neointimal hyperplasia after mechanical vascular injuries. Circ. Res. 93:783-790.
36. Campbell, J. H., Han, C. L., and Campbell, G. R. 2001. Neointimal formation by circulating bone marrow cells. Ann. N.Y. Acad. Sci. 947:18-24.
37. Quaini, F., Urbanek, K., Beltrami, A. P., Finato, N., Beltrami, C. A., Nadal-Ginard, B., Kajstura, J., Leri, A., and Anversa, P. 2002. Chimerism of the transplanted heart. N Eng J Med 346:5-15.
38. Glaser, R., Lu, M. M., Narula, N., and Epstein, J. A. 2002. Smooth muscle cells, but not myocytes, of host origin in transplanted human hearts. Circulation 106:17-19.
39. Bentzon, J. F., Weile, C., Sondergaard, C. S., Hindkjaer, J., Kassem, M., and Falk, E. 2006. Smooth muscle cells in atherosclerosis originate from the local vessel wall and not circulating progenitor cells in ApoE knockout mice. Arterioscler Thromb Vasc Biol 26:2696-2702.
40. Xu, Q. 2006. The impact of progenitor cells in atherosclerosis. Nat Clin. Pract. Cardiovasc. Med. 3:94-101.
41. O'Brien, E. R., Alpers, C. E., Stewart, D. K., Ferguson, M., Tran, N., Gordon, D., Benditt, E. P., Hinohara, T., Simpson, J. B., and Schwartz, S. M. 1993. Proliferation in primary and restenotic coronary atherectomy tissue: Implications for anti-proliferative therapy. Circ Res 73:223-231.
42. Iakovou, I., Schmidt, T., Bonizzoni, E., Ge, L., Sangiorgi, G. M., Stankovic, G., Airoldi, F., Chieffo, A., Montorfano, M., Carlino, M. et al 2005. Incidence, predictors, and outcome of thrombosis after successful implantation of drug-eluting stents. JAMA 293:2126-2130.
43. Finn, A. V., Nakazawa, G., Joner, M., Kolodgie, F. D., Mont, E. K., Gold, H. K., and Virmani, R. 2007. Vascular responses to drug eluting stents: importance of delayed healing. Arterioscler. Thromb. Vasc. Biol. 27:1500-1510.
44. Farb, A., Burke, A. P., Kolodgie, F. D., and Virmani, R. 2003. Pathological mechanisms of fatal late coronary stent thrombosis in humans. Circulation 108:1701-1706.
45. Grewe, P. H., Deneke, T., Machraoui, A., Barmeyer, J., and Muller, K.-P. 2000. Acute and chronic tissue response to coronary stent implantation: Pathologic findings in human specimen. J Am Coll Cardiol 35:157-163.
46. Finn, A. V., Joner, M., Nakazawa, G., Kolodgie, F., Newell, J., John, M. C., Gold, H. K., and Virmani, R. 2007. Pathological correlates of late drug-eluting stent thrombosis: strut coverage as a marker of endothelialization. Circulation 115:2435-2441.
47. Walter, D. H., Zeiher, A. M., and Dimmeler, S. 2004. Effects of statins on endothelium and their contribution to neovascularization by mobilization of endothelial progenitor cells. Coron. Artery Dis. 15:235-242.
48. Van, B. E., Tio, F. O., Couffinhal, T., Maillard, L., Passeri, J., and Isner, J. M. 1997. Stent endothelialization. Time course, impact of local catheter delivery, feasibility of recombinant protein administration, and response to cytokine expedition. Circulation 95:438-448.
49. Van, B. E., Bauters, C., Asahara, T., and Isner, J. M. 1998. Endothelial regrowth after arterial injury: from vascular repair to therapeutics. Cardiovasc Res. 38:54-68.
50. Joner, M., Finn, A. V., Farb, A., Mont, E. K., Kolodgie, F. D., Ladich, E., Kutys, R., Skorija, K., Gold, H. K., and Virmani, R. 2006. Pathology of drug-eluting stents in humans: delayed healing and late thrombotic risk. J. Am. Coll. Cardiol. 48:193-202.
51. Klugherz, B. D., Llanos, G., Lieuallen, W., Kopia, G. A., Papandreou, G., Narayan, P., Sasseen, B., Adelman, S. J., Falotico, R., and Wilensky, R. L. 2002. Twenty-eight-day efficacy and pharmacokinetics of the sirolimus-eluting stent. Coronary Artery Disease 13:183-188.
52. Farb, A., Heller, P. F., Shroff, S., Cheng, L., Kolodgie, F. D., Carter, A. J., Scott, D. S., Froehlich, J., and Virmani, R. 2001. Pathological analysis of local delivery of paclitaxel via a polymer-coated stent. Circulation 104:473-479.
53. Noble, W., Planel, E., Zehr, C., Olm, V., Meyerson, J., Suleman, F., Gaynor, K., Wang, L., LaFrancois, J., Feinstein, B. et al 2005. Inhibition of glycogen synthase kinase-3 by lithium correlates with reduced tauopathy and degeneration in vivo. Proc. Natl. Acad. Sci. U.S. A 102:6990-6995.
54. Bhat, R., Xue, Y., Berg, S., Hellberg, S., Ormo, M., Nilsson, Y., Radesater, A. C., Jerning, E., Markgren, P. O., Borgegard, T. et al 2003. Structural insights and biological effects of glycogen synthase kinase 3-specific inhibitor AR-A014418. J Biol. Chem. 278:45937-45945.

What is claimed is:

1. A method of treating or preventing vascular disease in a subject, comprising administering to said subject a device having a kinase inhibitor.

2. The method of claim 1, wherein the device is selected from the group consisting of a stent and a catheter.

3. The method of claim 1, wherein the vascular disease is selected from the group consisting of coronary artery disease and ischemic heart disease.

4. The method of claim 1, wherein the kinase inhibitor is a glycogen synthase kinase (GSK) inhibitor.

5. The method of claim 4, wherein the glycogen synthase kinase inhibitor is a GSK-3β inhibitor.

6. The method of claim 1, wherein the subject is a mammal.

7. The method of claim 6, wherein the mammal is a human.

8. The method of claim 1, wherein vascular repair is promoted.

9. The method of claim 1, wherein the kinase inhibitor is coated on the device.

10. The method of claim 1, wherein the kinase inhibitor is integrated in the device.

11. A device for treating or preventing vascular disease in a subject, said device comprising a kinase inhibitor.

12. The device of claim 11, which is selected from the group consisting of a stent and a catheter.

13. The device of claim 11, wherein the kinase inhibitor is a glycogen synthase kinase (GSK) inhibitor.

14. The device of claim 13, wherein the glycogen synthase kinase inhibitor is GSK-3β.

15. The device of claim 11, wherein the vascular disease is selected from the group consisting of coronary artery disease and ischemic heart disease.

16. The device of claim 11, wherein the subject is a mammal.

17. The device of claim 16, wherein the mammal is a human.

18. The device of claim 11, wherein vascular repair is promoted.

19. The device of claim 11, wherein the kinase inhibitor is coated thereon.

20. The device of claim 11, wherein the kinase inhibitor is integrated therein.


SEQUENCE LISTING (13 sequences)

SEQ ID NO: 1 - DNA, 24 bp, artificial sequence
  GAPDH fwd primer
cgcctggagaaagctgctaagtat

SEQ ID NO: 2 - DNA, 24 bp, artificial
  GAPDH rev primer
gcttcacaaagtggtcattgaggg

SEQ ID NO: 3 - DNA, 24 bp, artificial
  VLA1 fwd primer
acaagtgacagcgaagaacctcct

SEQ ID NO: 4 - DNA, 24 bp, artificial
  VLA1 rev primer
tgggtacagcacagggtaaccatt

SEQ ID NO: 5 - DNA, 24 bp, artificial
  VLA2 fwd primer
actttatctccagcggtacaaagt

SEQ ID NO: 6 - DNA, 24 bp, artificial
  VLA2 rev primer
tgggccttatcccaatctgaccaa

SEQ ID NO: 7 - DNA, 24 bp, artificial
  VLA3 fwd primer
caaagacaggcaaacggcaacgta

SEQ ID NO: 8 - DNA, 24 bp, artificial
  VLA3 rev primer
ttattggtcgcggtgagaagccta

SEQ ID NO: 9 - DNA, 24 bp, artificial
  VLA4 fwd primer
agggcaaggaagttccaggttaca

SEQ ID NO: 10 - DNA, 24 bp, artificial
  VLA4 rev primer
acatgaggaccaaggtggtaagca

SEQ ID NO: 11 - DNA, 24 bp, artificial
  VLA5 fwd primer
tgcctgagtcctcccaatttcaga

SEQ ID NO: 12 - DNA, 24 bp, artificial
  VLA5 rev primer
acatgaggaccaaggtggtaagca

SEQ ID NO: 13 - DNA, 26 bp, artificial
  modified labelled VLA4 probe
agcatttatgcggaaagatgtgcggg